(19) United States
(12) Patent Application Publication
(10) Pub. No.: US 2016/0298067 A1
(43) Pub. Date: Oct. 13, 2016

(54) METHOD FOR PRODUCING BIOGAS FROM BIOMASS USING ANAEROBIC DIGESTION AND A CORRESPONDING REACTOR

(71) Applicant: BIOGTS OY, Jyväskylä (FI)

(72) Inventors: Mika RAUTIAINEN, Leppävesi (FI); Annimari LEHTOMÄKI, Petäjävesi (FI)

(73) Assignee: BIOGTS OY, Jyväskylä (FI)

(21) Appl. No.: 15/038,262

(22) PCT Filed: Sep. 11, 2014
(86) PCT No.: PCT/FI2014/050694

§ 371 Date: May 20, 2016

(30) Foreign Application Priority Data
Nov. 21, 2013 (FI) 20136161

(51) Int. Cl. C12M 1/107; C12M 1/06; C12P 5/02

(52) U.S. Cl. CPC C12M 21/04; C12P 5/023; C12M 27/06

(57) ABSTRACT

The invention relates to a method for producing biogas from biomass using anaerobic digestion wherein biomass is fed as input to a reaction space using mechanical feed equipment while simultaneously advancing biomass contained in the reaction space as plug flow. Biomass is mixed separately in each block in the reaction space divided into successive blocks for feeding biomass to block-specific microbial strains. The number of blocks is at least three each comprising its own main microbial strain. Biogas produced as the result of anaerobic digestion of biomass is recovered. The microbial strain of each block is fed in front of the corresponding block and, in at least two of these blocks, the feed is reject obtained from the block as high consistency stock. The invention also relates to a corresponding reactor.

### CROSS-REFERENCE TO RELATED APPLICATIONS

This application is a U.S. National Stage of International Patent Application No. PCT/FI2014/050694, filed Sep. 11, 2014, which claims benefit of Finland Patent Application No. 20136161, filed Nov. 21, 2103.

### TECHNICAL FIELD

The invention relates to a method for producing biogas from biomass using anaerobic digestion, in which method
biomass is fed as input to a reaction space using mechanical feed equipment while simultaneously advancing biomass in the reaction space in the horizontal direction as plug flow,
biomass is separately mixed in each block in the reaction space divided into successive blocks for feeding biomass to block-specific microbial strains and advancing biomass in the reaction space, which has at least three blocks each including a main microbial strain of its own,
biogas produced as the result of anaerobic digestion of biomass is recovered.

The invention also relates to a corresponding reactor.

### BACKGROUND OF THE INVENTION

The invention is related to production of biogas from biomass. Biogas production is a method for processing organic waste and a method for producing renewable energy. Biogas production is based on a biological process called anaerobic digestion, in which microbes digest organic material or biomass in oxygen-free conditions so that methane-containing biogas is produced as the end product. Anaerobic digestion is a multi-step process, in which several different microbes are involved in the different steps of the digestion chain as shown in FIG. 1. Digestion chains for disintegrating biomass can be described in a simplified way as follows:

1) Polysaccharides (hydrocarbons)→Sugars→Short-chain fatty acids, H₂, CO₂→CH₄, CO₂
2) Proteins→Peptides, amino acids→Short-chain fatty acids, H₂, CO₂→CH₄, CO₂
3) Lipids→Long-chain fatty acids→Short-chain fatty acids, H₂, CO₂→CH₄, CO₂.

For example, the digestion chain of cellulose contained in lignocellulose is as follows, described in steps:
1) Decomposition of cellulose to sugars via hydrolysis:

(C₆H₁₀O₅)ₙ+nH₂O→nC₆H₁₂O₆
2) Decomposition of glucose units to acetate via acid fermentation:

C₆H₁₂O₆+4H₂O→2CH₃COO⁻+2HCO₃⁻+4H⁺+4H₂
3) Decomposition of acetate to methane via methanogenesis:

2CH₃COO⁻+H₂O→CH₄+HCO₃⁻

4H₂+HCO₃⁻+H⁺→CH₄+3H₂O

In the different steps of the digestion chain, active microbes also have different optimal conditions. Biogas produced as the end product of anaerobic digestion can be utilised as renewable energy in power and/or heat production or as traffic fuel.

The traditional biogas technology is mainly designed for the processing of wet waste fractions, such as wastewater sludge and animal manure. In this case, the processing most often takes place in completely mixed vertical cylindrical tank reactors at low dry contents (most often <10%), i.e. at high water contents (>90%). The most significant problem related to this method is that more than 90% of the raw material contained in the reactor is water. Energy (biogas) cannot be produced from water; instead, heating large quantities of water consumes remarkable amounts of energy. In addition, when processing of dryer waste fractions is desired in this type of completely mixed reactor, the input must be diluted with a liquid. Liquid can also be recirculated back to the reactor; however, many problems, such as the inhibiting effect of disintegration products and nitrogen compounds accumulating in the recirculated liquid, are often associated with this. Furthermore, a problem related to the use of a completely mixed tank is that all microbial strains live in the same space in homogeneous conditions and thus, the reaction conditions must be optimised according to the slowest step of the digestion chain, i.e. methane generation. In this case, the action of active microbes is not optimal in the other steps of the digestion chain.

Biogas production technologies based on so-called dry fermentation processes have been developed for the processing of dryer waste fractions. These processes can be operated at notably higher dry contents compared to the traditional biogas technology. Thus, a notably higher energy yield per reactor volume can be achieved.

One method of implementing a biogas plant based on a dry process is a biogas reactor operated on the so-called plug-flow principle. A biogas reactor operated on the plug-flow principle is most often a horizontal tank reactor into which biomass is fed from one end of the reactor and the material processed is removed from the other end of the reactor. During the processing, the material thus passes through the horizontal reactor based on a plug-type flow. A biogas reactor operated on the plug-flow principle can be operated at remarkably higher dry contents compared to the traditional biogas processes (for example, at dry contents of 10% to 30%). The process thus enables a wide raw material base (a possibility to also process dryer materials), a higher energy yield per reactor volume and more compact reactor constructions (less water taking up the reactor volume, more organic matter to be digested per reactor volume). In addition, many materials that cause settling problems in the traditional biogas processes, such as pieces of plastic and paper and sand mixed with biogas, do not cause similar problems in a biogas reactor operated on the plug-flow principle.

A challenge in a biogas reactor operated on the plug-flow principle, particularly when operating the reactor at high dry contents, is the arrangement of mixing. By mixing the contents of the reactor, unobstructed access of microbes to the material to be disintegrated is ensured (“inoculation”) and accumulation of disintegration products in the immediate vicinity of microbes is prevented. According to FIG. 1, accumulation of disintegration products generating in the various steps of the digestion chain inhibits i.e. decelerates the disintegration work of microbes and generation of methane, or may also completely prevent these. Mixing of the contents of the reactor contributes to local “dilution” of disintegration products as well as their transfer to become available for other microbes.

In earlier biogas reactor techniques operated on the plug-flow principle, mixing is most often implemented using a mixing shaft disposed in the longitudinal direction of the reactor. Publication U.S. Pat. No. 7,659,108 B2 proposes a plug-flow reactor in which biomaterial contained in the reactor is mixed using mixing blades supported on one shaft. However, a problem with such a construction is that optimisation of conditions for each reaction is difficult. This is disadvantageous for the efficiency of microbial action, when the anaerobic digestion process includes several different reaction steps, as shown in FIG. 1.

Publication US 2010/0062482 A1 proposing a horizontal tubular reactor for producing biogas is also known as prior art. In the reactor, part of biomaterial and water that has passed through the reactor is returned back to the initial part of the reactor from the end of the reactor. In the final part of the reactor, methanogenic microbes are dominant in the microbial population instead of the microbes of the hydrolysis step, which are specifically needed in the initial part of the reactor for providing an efficient digestion reaction. Hence, inoculation of the initial part of the reactor with digestate removed from the end of the reactor is not an optimal alternative for providing an efficient digestion reaction.

### SUMMARY OF THE INVENTION

An object of the invention is to provide a method for producing biogas from biomass, the method being more efficient and faster than prior art methods.

The method according to the invention is characterized by a method for producing biogas from biomass using anaerobic digestion, wherein the method includes: feeding the biomass as input to a reaction space using mechanical feed equipment while simultaneously advancing the biomass contained in the reaction space horizontally as plug flow, mixing the biomass separately in each block in the reaction space divided into successive blocks for feeding the biomass to block-specific microbial strains and transferring the biomass forward in the reaction space which includes at least three blocks each comprising a block's own main microbial strain, feeding the microbial strain of each block in front of such block and, at least in two of these blocks, the microbial strain fed to the block is reject obtained from the block as high consistency stock, and recovering biogas produced as a result of anaerobic digestion of said biomass.

Another object of the invention is to provide a reactor for producing biogases from biomass, the reactor being more efficient than prior art reactors. The reactor according to an embodiment of the invention includes a reactor for producing biogas from biomass using anaerobic digestion, comprising a frame delimiting a reaction space for biomass, the frame being channel-like and horizontal for plug flow of biomass and the frame having at least three successive blocks comprising their microbial strains, mixing equipment for mixing biomass and feeding microbes to biomass as well as transferring biomass forward in the reaction space, the mixing equipment comprising a reject collection and feed system for collecting reject from the blocks and feeding it to at least three blocks as high consistency stock, recovery equipment for recovering generating biogas while microbes consume organic material of biomass.

The object of the method according to the invention can be achieved with a method for producing biogas from biomass using anaerobic digestion, in which method biomass is fed as input to a reaction space using mechanical feed equipment while simultaneously forwarding biomass in the reaction space in the horizontal direction as plug flow. Biomass is mixed separately in each block in the reaction space divided into successive blocks for feeding biomass to block-specific microbial strains and transferring biomass further in the reaction space, which has at least three blocks each including a main microbial strain of its own. Biogas generating as the result of anaerobic disintegration of biomass is recovered. The microbial strain of each block is fed in front of the corresponding block and, at least in two of these blocks, the supply is reject obtained from the block as high consistency stock. Using block-specific adjustment of conditions, each anaerobic digestion reaction can be performed in more optimal conditions than in prior art methods. In this context, “in front of” means the beginning of a block, i.e. the opposite direction relative to the travel direction of biomass. The location to which a microbial strain is fed determines the starting point of the block, since the microbial strain gains strength in this location in a remarkable way. The object of the invention is achieved, because the supply of the microbial strain in front of each block increases the concentration of the microbial strain and notably accelerates the growth of the microbial strain to its optimum in this block, which in turn improves the reactions provided by the microbes and thereby the production of biogas.

Advantageously, a microbial strain is recirculated inside the block as reject from the end to the beginning of the block. This recirculation transfers the concentrated microbial strain at the end of the block to the beginning of the block, wherein the microbial strain is inherently weak.

Advantageously, reject is fed through mixing equipment. The supply of reject through mixing equipment reduces friction between the mixing equipment and biomass and thus decreases the power consumption. In addition, biomass in each block can be inoculated with a concentrated microbial strain, which is needed in this block for promoting reactions. Inoculation of a correct microbial strain approximately at the centre of the reactor is technically very difficult using other methods. At the same time, part of the input can be fed through the mixing equipment approximately to the centre of the reactor, for example.

The dry content of the high consistency stock may range from 3% to 35%, advantageously from 10% to 20%. The biomass transferred as high consistency stock to the block preceding this block has an abundant content of the microbial strain of the block, which can thus be moved to the beginning of the block, where the microbial strain would be inherently weaker. A significant part of the microbial strain involved in the reactions of the anaerobic digestion chain mainly lives on the surface of solid material and not in liquid “reject”, which has been used as the “inoculant” in other applications.

Advantageously, reject is removed from the blocks as a bottoms product using a vacuum. The vacuum also enables the transfer of the high consistency stock. The discharge as a bottoms product enables utilising the hydrostatic pressure of the liquid present in the reaction space for transfer.

The vacuum is advantageously generated using a single pump and applied to a selected block of the valve system. By using a single pump, the reactor can be implemented at remarkably lower investment costs.

Reject can be fed backwards in front of the block, to the suction valves, at regular intervals via a reject collection and feed system for keeping the reject collection and feed system clean. This back feed can efficiently prevent plugging of the collection system. In other words, reject can sometimes be fed backwards via the discharge connector to the block from which the reject is taken.

The mixing equipment may consist of rotating blade elements, and the mixing equipment of the block located last in the travel direction of biomass is operated in the opposite direction compared to that of the mixing equipment of the other blocks. An opposed mixing direction facilitates the release of gas bubbles of biogas, generating in methanogenesis, from the solid biomass.

Advantageously, biomass is inoculated by rotating the mixing equipment backwards. In this context, “backwards” means that the mixing equipment is rotated in such a way that its force for moving biomass is applied towards the initial part of the reactor, from where at least the main part of biomass is fed to the reactor. By moving the biomass to be digested backwards in the reactor, it can be ensured that the microbial strain spreads to the raw material to be disintegrated and the disintegration products are moved away from the area around microbes.

According to an embodiment, liquid input is fed to the reactor via the mixing equipment. In this way, the input can be fed to a selected point in the reactor in accordance with the processing time required by the input. In other words, for example, the first block of the reactor can be excluded for an easily disintegrating raw material, reducing in this way the dwell time of biomass in the reactor.

Advantageously, the temperature of biomass is block-specifically adjusted in each block. Block-specific temperature adjustment enables more accurate optimisation of the conditions, which improves biogas production. In this context, temperature adjustment may mean either heating or cooling of biomass depending on the conditions. Advantageously, biomass supplied to the reactor requires heating, and heat can be recovered from the biomass discharged at the end of the reactor, i.e. biomass can be cooled, for preheating the raw material supplied, for example.

Inoculation and heating of each block can be independently monitored and controlled. In this way, it can be ensured that each anaerobic digestion reaction can take place in the conditions that are favourable for the reaction. With independent control, the blocks and thus, the reaction conditions as well, are at least almost independent of each other.

High consistency stock can be fed from the walls and/or the bottom of the reaction space for facilitating the flow of biomass. High consistency stock fed from the walls and/or the bottom reduces the force required for advancing the biomass to be disintegrated by reducing the friction between biomass and the inner surface of the reaction space. At the same time, the high consistency stock supplied inoculates the biomass contained in the reactor.

According to another embodiment, biogas can be fed from the walls and/or the bottom for facilitating the flow of biomass. Biogas efficiently detaches biomass from the walls and/or the bottom of the reaction space as well as separates biogas bubbles contained in biomass. This is significant particularly in the last block of the reactor since the method prevents the removal of biogas with the digestate.

The novel type of mixing in the method according to the invention is carried out by mixing the biomass contained in the reaction space of the reactor block-specifically using, for instance, mechanical horizontal or vertical mixers, the shafts of which are advantageously disposed transversely relative to the direction of the reactor. The number of mixers depends on the length of the reactor; generally, it is possible to use 3 to 10 mixers per one reactor. Several mixers can also be present in an individual reactor block. An advantage of this mixing method is that the reactor can be agitated block-specifically by moving the stock locally forward or backward. The operation of each mixer can be separately adjusted; i.e. the efficiency and direction of mixing as well as the temperature (between 20 and 55° C.) of the reactor are adjustable for each block. The conditions of the reactor (for example pH, temperature, gas production) can be monitored in real-time locally and block-specifically (sensors located in the area of each block), and the information obtained can be compared with the mixing operation and the reactor load. By moving the microbial strain of at least two blocks as high consistency reject backwards in the travel direction of biomass in front of the block, a sufficient population of the microbial strain is ensured in the entire block. The difference compared to earlier biogas reactors based on a longitudinal mixing shaft is that the microbial strain can be moved locally and block-specifically in the reactor, thereby locally increasing the active microbial strain in the anaerobic digestion step taking place in the area of this block. At the same time, the conditions of the reactor can be optimised block-specifically and the conditions can be locally optimised according to the optimum conditions of microbes involved in the different steps of the digestion chain. Thus, a better digestion result can be achieved and biogas production is maximised.

The object of the reactor according to the invention can be achieved with a reactor that includes a frame delimiting a reaction space for biomass, said frame being channel-like and horizontal for the plug flow of biomass and having at least three successive blocks including their microbial strains. In addition, the reactor includes mixing equipment for mixing biomass and feeding microbes into biomass as well as advancing biomass in the reaction space, and recovery equipment for recovering the biogas generated while the microbes consume the organic material of biomass. The mixing equipment includes a reject collection and feed system for collecting reject from the blocks and feeding it as high consistency stock to at least three blocks. With such a reactor, the operating conditions of microbes used in each anaerobic digestion reaction can be optimised by adjusting the condition of the block concerned. Optimised conditions improve the action of microbes and thereby accelerate the disintegration of biomass to the desired end product, i.e. methane. The reject collection and feed system enables feeding the concentrated microbial strain contained in the reject in front of the block as high consistency stock, which ensures a sufficient microbial strain immediately in the initial part of this block. By feeding reject to each block, the motor power required by the mixing equipment can also be reduced.

Advantageously, the mixing equipment is implemented using shafts disposed transversely relative to the longitudinal direction of the reactor frame for mixing biomass block-specifically. A transverse shaft enables independent mixing of each block regardless of the number of successive blocks.

Advantageously, the reject collection and feed system is arranged to feed reject as high consistency stock via the mixing equipment in order to reduce friction. At the same time, the microbial strain can be moved from the end to the initial part of the block or, if necessary, from one block to another. If necessary, the same feed equipment can be used to supply the reactor with input which is not useful when supplied to the initial part of the reactor, where the conditions are not optimal for the input supplied.

The reject collection and feed system may include a high consistency stock pump for feeding reject at a dry content of 3% to 35%, advantageously 10% to 20%. Thus, the reject contains a sufficient amount of solids, on the surface of which methanogens, i.e. the microbial strain that is responsible for the generation of methane, mainly live.

According to an embodiment, the supporting and operating elements of the mixing equipment of each block are located outside the frame. Thus, maintenance of the mixing equipment can be performed outside the reactor, which remarkably facilitates maintenance.

Advantageously, the reaction space is divided into blocks that are specific for the reaction steps of anaerobic digestion including at least a hydrolysis block, an acid fermentation block and a methanogenesis block. Thus, the conditions of each block can be optimised specifically for each block to optimise biogas production. Advantageously, the length of the hydrolysis block is 25% to 35% of the total length of the reaction space, the length of the acid fermentation block is also 25% to 35%, and the length of the methanogenesis block is 30% to 50% of the total length of the reaction space.

When referring to a block-specific microbial strain, it should be understood that each block has mixed populations of microbes of different blocks. In the hydrolysis block, the strain of microbes that are essential for hydrolysis comprises 50% to 95% of the number of all microbes, and the same applies to the acid fermentation block. The microbial strain of the methanogenesis block is more sensitive and therefore, the microbial strain comprises from 30% to 90% of the total microbial strain of this block.

Advantageously, the reactor also includes independent temperature control equipment for controlling the temperature of biomass separately in each block. In this way, the temperature of biomass can be more accurately adjusted, thus facilitating optimisation of conditions.

Advantageously, the frame includes a frame cage for supporting the blocks to each other. The frame cage ensures the overall rigidity of the reactor construction and the support required by the fastening points of the shafts of the mixing equipment.

Advantageously, the reactor includes equipment for independently monitoring and controlling the inoculation, mixing and heating of each block. In this way, each block can be controlled independently of the other blocks.

According to an embodiment, the reactor includes equipment for feeding biomass and/or biogas from the walls and/or the bottom of the reaction space into biomass for facilitating the flow. By feeding high consistency stock or biogas, the friction between the reactor frame and biomass can be reduced while simultaneously inoculating biomass.

Advantageously, the reactor frame includes sub-frames, which are identical with each other except for their lengths and heights. Such a construction makes the reactor affordable to manufacture.

Advantageously, a sub-frame includes planar modules, which are identical with each other in each sub-frame. A modular reactor can be easily packed in marine containers for transportation and is quickly to install and set in the operating conditions at the installation site.

Advantageously, the sub-frames form a direct flow channel, which serves as the reaction space. A channel-like construction enables the advancement of biomass as plug flow.

According to an embodiment, the high consistency stock pump is a hose pump. Such a pump is particularly suitable for pumping high consistency stock.

The number of blocks can be three, advantageously between three and six. Thus, there is at least one block for each main digestion reaction, and the conditions of each block can be optimised for each microbial strain.

In this context, when referring to blocks, it should be understood that the blocks proposed in this application are reactor components, each of which having its own main microbial strain and, advantageously, being independently controlled. An individual block can include one or more modular sub-frames and pieces of mixing equipment. The boundaries of the blocks can vary according to the reaction areas generated by the biomass supplied. Advantageously, a block boundary means the area in which the main population of the microbial strain changes from one population to another. Excluding the gas space, there are advantageously no mechanical limitations, such as intermediate walls or equivalent, between the blocks; instead, the biomass supplied can pass through the reactor unobstructed passing through different blocks. The conditions in each block are advantageously different. In addition, when referring to biomass, it should be understood that it means the raw material that is supplied to the reaction space and anaerobically digested there, whereas digestate and reject mean the high consistency stock produced as the end product of anaerobic digestion, which is discharged from the reaction space.

The novel construction of the reactor according to the invention is advantageously based on prefabricated modules. Modules mean the concrete plate-like components of the frame, which are combined to form channel-like sub-frames which, when placed successively, form the frame of the reactor. Advantages of a biogas plant based on prefabricated modules include, for example, that the size of the plant is easily scalable (by increasing the number and length of the sub-frames; i.e. by adjusting the dimensions of the reactor), the plant can be quickly installed and taken into use at the application site (compared to traditional biogas plant solutions, which are often cast in cement on site, for example), and the manufacture of modules with standard dimensions enables utilising serial work, which reduces manufacturing costs. Furthermore, modules enable easy transportation of the reactor using normal marine containers. In this context, when referring to sub-frames, the concrete construction of the frame is meant, wherein the modules form a reaction space within the sub-frame, whereas, when referring to blocks, a construction that is independent in terms of adjustment and control is meant, and this can be composed of one or more sub-frames.

A reactor according to the invention is capable of disintegrating biomass in a quantity corresponding approximately up to 9 kg of organic matter per a volume corresponding to one reactor cubic meter in a day (9 kg VS/m³/d). This quantity may vary remarkably depending on the characteristics of the input. The result is up to five-fold in proportion to prior art reactors. Thus, the benefit from the method according to the invention can be seized, for example, by manufacturing a reactor that is notably smaller than would be required by prior art methods.

### BRIEF DESCRIPTION OF THE DRAWINGS

The invention is described below in detail by making reference to the appended drawings illustrating some of the embodiments of the invention, in which

FIG. 1 is a basic view illustrating anaerobic digestion of biomass from raw material to an end product,

FIG. 2 is a lateral cross-sectional basic view of the reactor according to the invention,

FIG. 3 is a basic cross-sectional end view of the reactor according to the invention,

FIG. 4 is a lateral basic view of the reactor according to the invention,

FIG. 5 is a process chart of an embodiment of the reactor according to the invention.

### DETAILED DESCRIPTION OF THE INVENTION

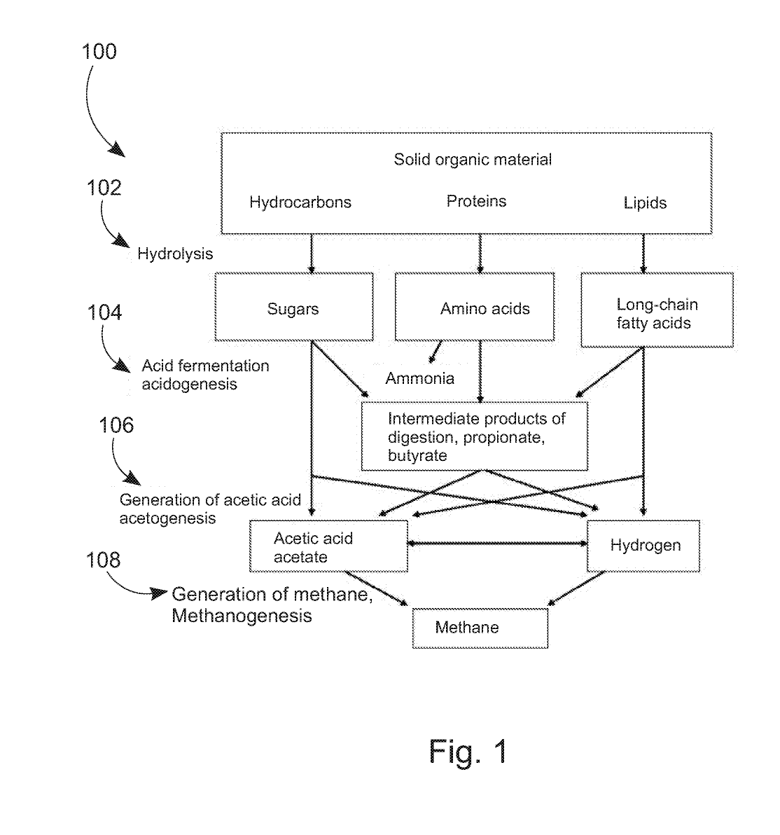

According to FIG. 1, the process of anaerobic digestion includes several steps 100, in which microbes disintegrate organic matter. Since each reaction is best carried out in optimum conditions for the reaction concerned, efficient utilisation of anaerobic digestion in biogas production is greatly dependent on the optimisation of the various sub-processes. In conditions that are favourable for the hydrolysis of polysaccharides 102 and fermentation, the pH range is approximately 6.5 to 7. In conditions that are favourable for the fermentation of sugars, i.e. acid fermentation 104, the pH range is approximately 5 to 6. In conditions that are favourable for the generation of acetic acid 106, the pH range is approximately 6.5 to 7.5. In conditions that are favourable for the generation of methane 108, i.e. methanogenesis, the pH range is approximately 6.5 to 8.0. The microbial strain essential for methanogenesis is killed if the pH is below 6. In addition, for the hydrolysis step, it is useful if the oxygen content of stock is low, whereas oxygen is extremely toxic for the microbes of the methanogenesis step. In addition, the microbes involved in the methanogenesis and acetogenesis steps are very sensitive to accumulation of inhibition agents (e.g. short-chain fatty acids, ammonia). For example, the temperature range used in the process may be 35-37° C. or approximately 55° C. However, the temperature may vary according to the input and the microbial strain used. Since the composition of biomass used as raw material in the method may vary remarkably, the reactions included in anaerobic digestion can also vary.

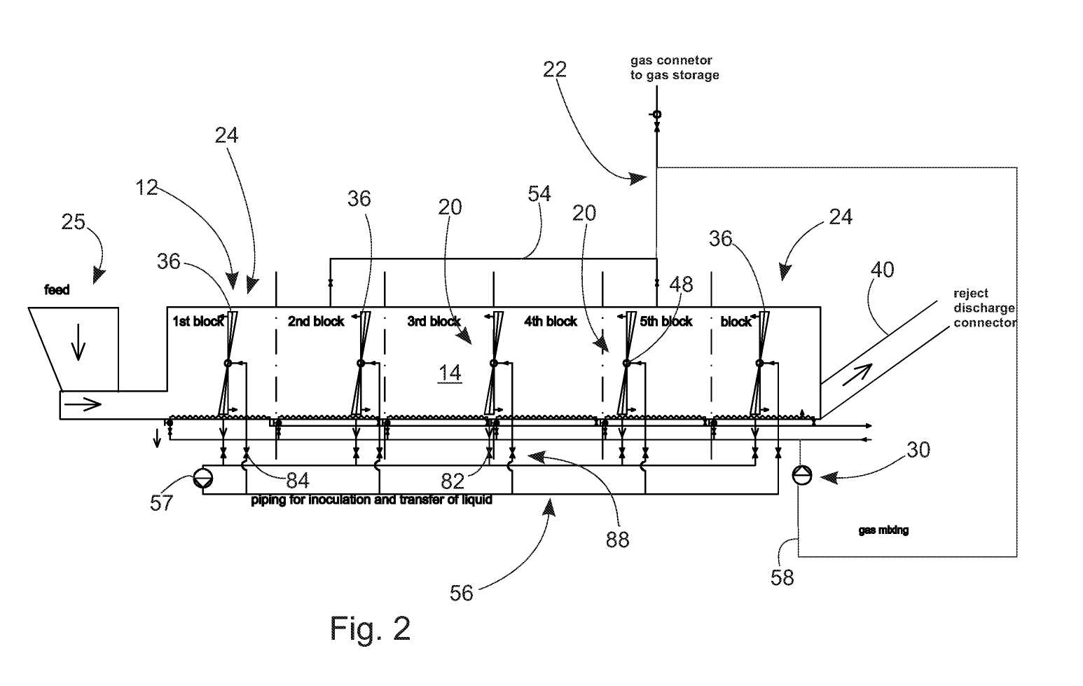

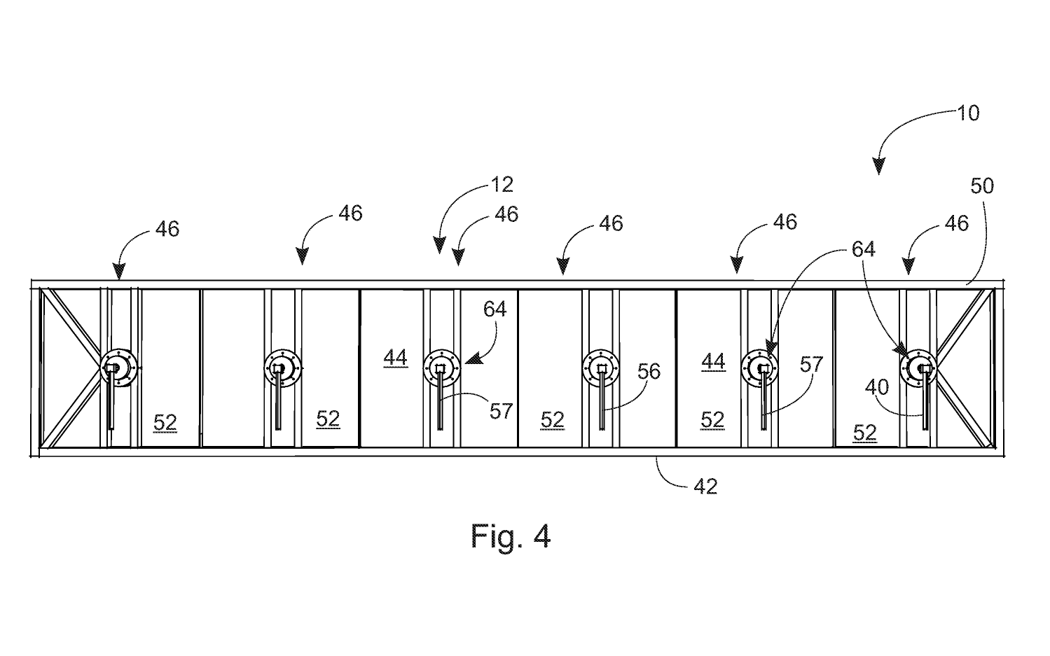

Below is a more detailed description of the construction of the reactor according to the invention. According to FIG. 2, the reactor 10 according to the invention is composed of a modular frame 12. More precisely, the modular frame 12 advantageously includes 3 to 10 sub-frames 46, which form the channel-like frame 12 of the reactor in the horizontal plane delimiting a reaction space 14 within, wherein biomass 16 is disintegrated to biogas and digestate, via anaerobic digestion. If necessary, the number of sub-frames can be notably greater than 10. The sub-frames 46 are channel-like constructions with equal diameters and shapes, and only the lengths of the sub-frames 46 may vary in the travel direction of biomass. For example, the width of the sub-frames may be 2.2 metres, in which case the heights and lengths of the sub-frames only vary according to the volume required of the reactor. In this context, the longitudinal direction of the reactor 10 means the same direction as the travel direction of biomass 16 advancing as plug flow in the reaction space. The modules forming the sub-frames can be prefabricated, surface-finished and insulated. Advantageously, the sub-frames 46 are locked in place with an external beam construction 50 and sealed against each other with seals, as shown in FIG. 4. The beam construction 50 locks the sub-frames 46 to form a continuous frame 12 of the reactor 10. The shape of the sub-frames 46 can be a square, for example, and the cross-section of the reaction space 14 delimited by these can also be a quadrangle or a square. On the other hand, the cross-section of the reaction space can also be any other form; however, a quadrangle and its variations are the simplest in terms of technical implementation.

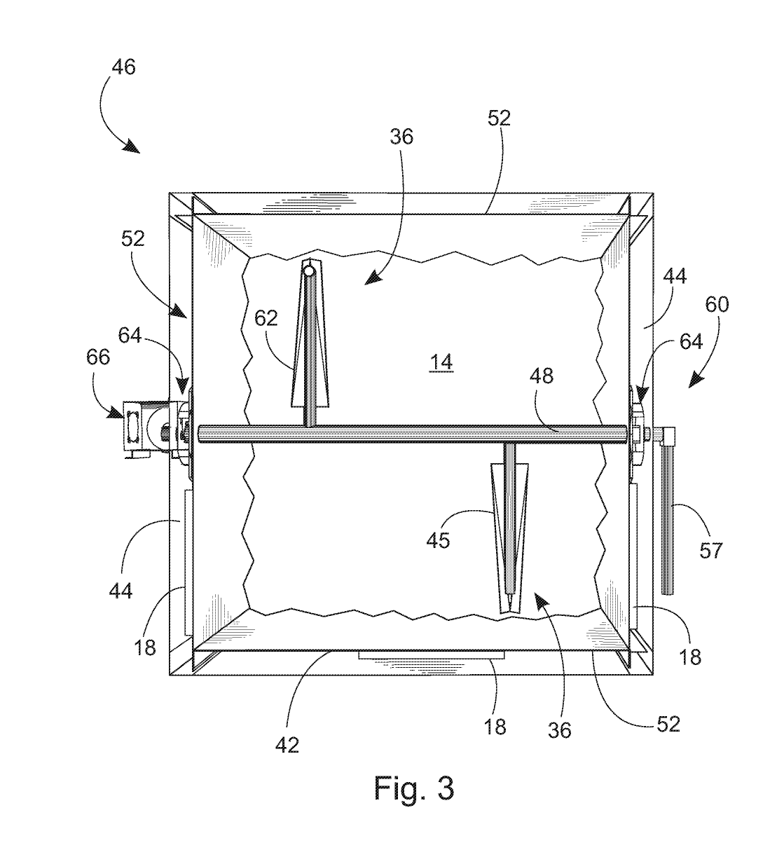

In addition to the frame 12, the reactor 10 includes mixing equipment 20, which is used to mix the biomass 16, serving as raw material, contained within the frame 12. According to the invention, each block 24 advantageously includes mixing equipment 20 of its own, which may consist of blade mixers 36, as shown in FIG. 3, supported by a shaft 48 disposed transversely relative to the longitudinal direction of the reactor 10 through the sub-frame 46. A block 24 refers to a controlled and adjusted unit of one or more sub-frames 46 wherein the conditions can be adjusted to suit the microbial action of the main microbe strain dominant in the area of the block. According to an advantageous embodiment of the invention, each block 24 includes at least one blade mixer 36 of its own, which enables controlling the mixing of biomass separately for each block. Alternatively, instead of a blade mixer, a mixing screw or an equivalent mechanical device can be employed, which can be used to move biomass to different directions in the reaction space. According to FIG. 2, the number of blade mixers 36 may be equal with the number of sub-frames 46. Thus, the number of blocks 24 can also be equal.

FIG. 3 is a cross-sectional end view of a reactor according to the invention. Advantageously, the supporting of the mixing equipment 20 can be arranged in the reactor 10 in connection with the beam frame. In this case, the drive and gear 66 of each blade mixer 36 and the bearings 64 of the shafts 48 are located outside the frame 12 of the reactor 10. This considerably facilitates maintenance of the mixing equipment 20.

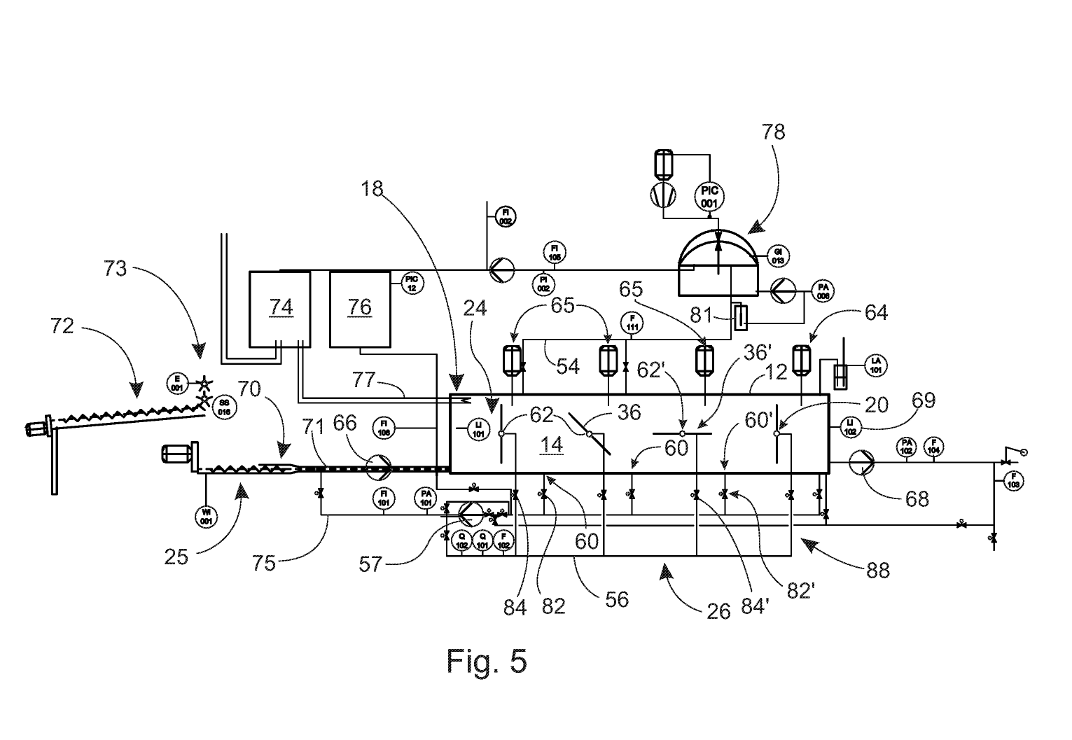

The reactor 10 may also include temperature adjustment equipment 18 (shown in FIG. 5) for adjusting the temperature of biomass 16 to a temperature that is optimal for the microbial action. Using mixing and temperature control equipment that is independent relative to the blocks, the conditions and mixing can be made optimal for the microbial action in each block. For example, the temperature control equipment 18 may consist of resistances installed in the modules 52 of the sub-frame, which are used to heat the constructions of the sub-frame 46 and thereby the biomass 16. Heating is important for the first two blocks, whereas in the block or blocks following these, in which methanogenesis takes place, it is no longer absolutely necessary to heat biomass, or it can be even cooled without noticeably affecting the yield of biogas. Cooling can be carried out using heat exchangers included in the temperature control equipment, which may preheat the biomass supplied to the reactor, for example. Gas boilers can also be used for heating to heat the water used in the heating circulation system, which can be advantageously controlled in three or more block-specific circuits.

To recover biogas obtained as the product, the reactor 10 includes recovery equipment 22 for collecting biogas from the reaction space 14. The recovery equipment 22 may consist of a pipework 54, shown in FIG. 2, constructed in the upper part of the frame 54, for recovering the generating biogas in a storage tank or equivalent. In addition, the reactor 10 may include mechanical feed equipment 25 for feeding biomass 16 into the sub-frames 46. The feed equipment may consist of a screw conveyor or equivalent, which feeds the biomaterial into the first sub-frame. According to an alternative, the reactor may include a feed funnel, through which the raw material is supplied to the reactor.

In the reactor according to the invention, the mixing equipment 20 includes a reject collection and feed system 56 of FIG. 2. The reject collection and feed system 56 is arranged in the lower part of the sub-frames 46 and the collection and feed system 56 is used to collect reject from the disintegrating biomass 16 to be fed in front of the block 24. The collection and feed system is not shown in FIG. 3; however, it should be understood that the sub-frames include such equipment. In this context, reject means high consistency stock. The dry content of high consistency stock ranges from 3% to 35%, advantageously from 10% to 20%. On the other hand, stock at a dry content ranging from 3% to 5% can also be called low consistency stock in some contexts. According to FIG. 2, the collection and feed system 56 advantageously includes a single high consistency stock pump 57, which is used to transfer the reject within the pipework. A valve system 88 arranged in the pipework opens valves 82 and 84 of the desired block 24 and closes valves 82 and 84 of the other blocks 24, in accordance with the control of the control system of the reactor 10. Advantageously, reject supply in front of a block means the supply of reject to the feed connector preceding the reject discharge connector; however, in some cases, reject can also be supplied even to a feed connector located earlier relative to the feed connector preceding the discharge connector. Advantageously, reject is sucked from the bottom of the reactor, because the liquid present in the reactor inherently creates a liquid pressure, which facilitates the transfer of reject.

Since the collection of reject is advantageously a part of inoculation, it must be ensured that the reject collection and feed system is kept clean and in good operating conditions. For this purpose, reject can sometimes be fed backwards via the discharge connector to the block from which reject is taken. In this way, plugging of the pipework is prevented. For example, a cleaning supply can be carried out two times a day or when an obstruction is detected on the suction side of the pump or pumps (the automation system monitors the flow rate and automatically tries to remove the obstruction using a counterflow).

Reject removed from a block can be supplied at a high pressure along the pipework 40 shown in FIG. 2 to the channel located within the hollow shafts 48 of the blade mixers 36 and further to the blades 45 of the blade mixer 36. In this context, high pressure means a pressure of 0.2 to 20 bar. The blades can include nozzles through which reject is supplied into biomaterial when using blade mixers. Alternatively, instead of or in addition to reject, liquid biomass can be fed through the mixing equipment as input.

According to an embodiment, the reactor also includes equipment 30 shown in FIG. 2 for reducing friction between biomass and the biomass contained in the reaction space, said equipment 30 including equipment 58 according to FIG. 2 for feeding high consistency stock and/or biogas from the walls 44 or the bottom 42 of the sub-frame 46 of FIG. 4 into the sub-frame 46 using a pump, for example. The high consistency stock and/or biogas supplied reduces the friction between the biomass 16 contained in the sub-frame 46 and the module 52, which in turn decreases the power requirement of the mixing equipment 20. High consistency stock and/or biogas can be supplied in a pointwise manner, in which case high consistency stock and/or biogas supplied into biomass displaces biomass and forms an opening therein, thus improving the advancement of biomass to be disintegrated in the reaction space. Advantageously, the high consistency stock is biomass reject. While the high consistency stock and/or biogas reduces the friction between biomass and the reactor frame, it also simultaneously inoculates the reactor. In addition, the mixing of liquid/gas “releases” gas that is bound to solids and ensures that methane is not removed with the reject.

In this context, it should be understood that in addition to providing mixing, the mixing equipment, such as blade mixers, also functions as the main element to push biomass further as plug flow. According to an embodiment, the inner surface of the frame can be coated with a coating equivalent to a Teflon coating, for example, which reduces the friction between biomaterial and the frame and prevents biomaterial from attaching to the inner surface of the frame.

Advantageously, the reactor according to the invention includes a considerable number of measuring sensors, which monitor the conditions of each block in real-time. Parameters to be measured include at least the pH and temperature in each block as well as the overall gas production of the reactor. Based on these, separate control parameters are formed at least for the mixing equipment, temperature control equipment and reject supply, specifically for each block. Advantageously, a control parameter is also established for the equipment for reducing friction, based on the same criteria. The amount of organic matter contained in biomass may also be an object of measurement.

In the method according to the invention, a major part of the reaction space is filled with liquid and biomass so that new raw material supplied to the reaction space, i.e. biomass to be disintegrated, is fed below the liquid level. This guarantees that air cannot enter the reaction space with biomass, which would destroy the microbial strain carrying out anaerobic digestion. Although the liquid level and biomass are not shown in FIG. 3 for the sake of clarity, it should be understood that the reaction space is filled with biomass almost up to the ceiling and the liquid level extends to a distance of about 20 cm from the reactor ceiling. The removal of digestate is controlled in the reactor in such a way that the liquid is always kept at the level required. The microbial strain itself can be transferred to the reaction space during the activation of the reactor from another reactor, for example. The biomass supplied can be biodegradable biomass generated in communities, agriculture or industry, such as animal manure, bio-waste produced by households, restaurants, trade or the food industry, sludge from wastewater cleaning, plant biomass or equivalent; however, not material with a high lignin content, such as wood pulp.

Advantageously, the dry content of stock in the reaction space can range from 10% to 35%, but materials dryer than this are difficult to mix. The dry content decreases towards the end of the reactor where disintegration has proceeded further. Biomass can be supplied to the reaction space using a screw feeder, for example. For example, feeding can take place at intervals of an hour, 24 hours a day, depending on the quantity of organic matter and biodegradability of biomass used as raw material.

In the method according to the invention, according to FIG. 2, biomass 16 can be inoculated in four different ways: by rotating the mixing equipment 20, by feeding reject with the reject collection and feed system 56 through the mixing equipment 20, by feeding high consistency stock and/or biogas from the sides and/or the bottom of the frame 12 of the reactor 10 using equipment 30 for reducing friction, or by adding reject to the input already before feeding it to the reactor. When anaerobic digestion reactions are in process in the reaction space, the gas production, pH and temperature are continuously monitored, whereas inoculation and mixing are advantageously intermittent for energy saving purposes. As a consequence of these changes, the mixing and heating of the reactor, the biomass supply and the reject supply are controlled by the reject collection and feed system. For example, if it is detected that the pH drops to an insufficient level or gas production decreases in the area of a block, the conditions can be locally influenced by improving mixing in this block and by increasing or decreasing the reject supply to this block.

The purpose of the mixing equipment 20 is to advance biomass 16 in the reaction space 14 and to mix biomass 16 so that the microbes receive fresh nutrition. If mixing is not performed frequently enough, a layer of disintegration products, which may inhibit the microbial action, is generated around microbes. For example, mixing can be performed for 15 minutes at intervals of an hour while simultaneously feeding reject to the block. Advantageously, in addition to the mixing direction providing the advancement force, mixing is carried out in the opposite direction, which provides a different type of mixing. For example, when using a blade mixer, parallel mixing always moves biomass in a certain place to a certain direction relative to the blade mixer. A change in the mixing direction adds different mixing directions, which improves biomass mixing, microbial inoculation and acquisition of organic raw material for microbes. Generally, the pass through of biomass in a reactor may be 11 to 50 days depending on the biomass used as raw material.

Mixing and its direction are determined according to the values measured; however, backward mixing is generally performed slightly before starting the mixing that advances biomass. The efficiency of mixing varies for each block in the reaction space. In the last block, mixing is advantageously the most efficient to allow separating even the rest of biogases, which may exist as bubbles within solid digestate in so-called gas pockets, from the digestate exiting the reaction space. This is important to achieve efficient biogas recovery and to prevent escape of methane with digestate to the atmosphere, wherein it is a strong greenhouse gas. Advantageously, in the last block, mixing equipment is rotated to the opposite direction compared to the other blocks, for improving mixing. Digestate removed from the reactor can be delivered to separation, wherein liquid is separated from it. This liquid can be used for cleaning the reject collection and feed system, for example.

In the oxygen-free conditions of the reaction space, the microbial action provides anaerobic digestion of biomass, which, according to the prior art technology, includes the hydrolysis, acid fermentation (acidogenesis), acetic acid generation (acetogenesis) and methane generation (methanogenesis) steps. The individual steps and the related reactions take place gradually and partly overlapping each other in the reaction space. Advantageously, hydrolysis and acidogenesis take place mainly in the initial part of the reaction space, whereas acetogenesis and methanogenesis occur mainly at the end of the reaction space. As the consequence of the reactions, biogas containing about 50%-75% (v/v) of methane (CH₄) can be produced from biomass as the end product, while the rest mainly consists of carbon dioxide (CO₂). In addition to these, the end product may contain small amounts of other gases and impurities, such as 100-3,000 ppm of hydrogen sulphide (H₂S). Depending on the end use of biogas, biogas obtained with the method can be purified to remove carbon dioxide, if biogas is used, for example, as fuel in the road traffic use. On the other hand, if biogas is used in combustion boilers for energy and district heat production, it can be used as such. As a consequence of disintegration reactions, 50% to 90% of the organic matter contained in the input is converted to biogas and liquid in the reaction space. If necessary, digestate generating as a by-product, may be dried or processed further in other ways and utilised for fertilisation purposes or as a soil conditioner, for example.

The dimensions of a reactor according to the invention can remarkably vary depending on the application. For example, the size of the reactor may be 0.5 m×0.5 m×1.5 m; however, it is scalable to a size class of 12 m×12 m×36 m or larger. In connection with large reactor sizes, several feed devices can be used to achieve uniform supply. Here, 12 m refers to the height and width of the reactor and 36 m refers to the length of the reactor in the longitudinal direction of the reaction space.

The control of a reactor according to the invention can be implemented, for example, using a conventional PC as a user platform, on which the control software of the reactor is run. A field bus for the data transfer is provided between the PC and actuators, sensors and other devices, such as valves, required for the control. A method according to the invention can be fully automated, in which case the software controls the operation of the reactor based on preselected criteria in compliance with preselected rules.

FIG. 5 shows a reactor according to an embodiment of the invention as a process chart together with auxiliary equipment associated with the reactor. In this embodiment, the process is started at a feed table 72 to which solid biomass is advantageously supplied as bales, which are broken into smaller chops with a bale breaker 73. From the bale breaker 73, the input drops to a feed screw 70, which supplies the input to the reactor 10 via the feed connector 71 at regular pre-set intervals, for example, once an hour. A crusher pipe, which breaks down the input further, may be located in the feed connector 71. In addition to solid biomass, the feed connector 71 can be supplied with the liquid reject of the reactor through the line 75, for reducing friction. The reactor 10 can also be supplied with liquid input, such as lipids, which can be stored in the tank 76. Liquid input can be delivered to the reactor via the high consistency stock pump 57 of the reject collection and feed system 56.

According to FIG. 5, the reactor 10 may include four mechanical mixers, which can be blade mixers 36 in this connection. Each blade mixer 36 is advantageously provided with a motor 65 and frequency converter of its own, which can be used to adjust the speed of rotation. For example, the output of an individual motor can be 4 kW and the speed of rotation 6 revolutions per minute. The frame 12 of the reactor 10 is divided into at least three blocks 24; i.e. a hydrolysis block, an acid fermentation block and a methanogenesis block. In each block 24, reject is supplied in front of the block using the reject collection and feed system 56. According to FIG. 5, reject is advantageously removed from the block 24 via the discharge connector 60 and supplied to the collection and feed system 56 as high consistency stock utilising a vacuum generated by the high consistency stock pump 57. For example, at the third blade mixer 36′, reject is removed via the discharge connector 60′ when the discharge valve 82′ is open. Reject passes via the high consistency stock pump 57 and is supplied to the reject feed connector 62′ via the open feed valve 84′. In this case, the reject feed connector 62′ is advantageously located on the blades of the blade mixer 36′. Generally, reference number 62 refers to a feed connector, reference number 82 to a discharge valve and reference number 84 to a feed valve.

In other words, the microbial strain of each block is advantageously recirculated within the block so that the microbial strain is removed from the block as reject and advantageously sent back to the block via the mixing equipment. In each block, the reject discharge connector is located at a distance from the mixing equipment or the reject feed connector from which reject is sent back to the block. This distance varies according to the scale of the reactor; however, the feed connector and the reject discharge connector are advantageously located at a distance corresponding to 0.2 to 0.6 times the block length, the reject discharge connector being located as close as possible to the end of the block. The distance enables the microbial strain to develop naturally within the block.

Disintegrated biomass or digestate is removed from the end of the reactor 10, i.e. the last block 24, using a pump 68. The pump is used, based on the measurement of the liquid level meter 69 of the reactor 10, when the preselected liquid level is exceeded. Digestate removed is advantageously delivered to a dry digestate storage where the dry matter and liquid are separated using a matrix pipe. Biogas generating in the reactor can be recovered in a gas storage 78. Advantageously, a condensate well 81 is also provided in connection with the gas storage, to which water condensing from biogas at 100% moisture is collected. Part of biogas can be used for heating the liquid heating circuit 77 of the reactor with a gas boiler 74.

1-20. (canceled)

21. A method for producing biogas from biomass using anaerobic digestion, comprising:
feeding biomass, using mechanical feed equipment, as input to a horizontal reaction space that is divided into at least three successive blocks containing block-specific microbial strains while simultaneously advancing biomass contained in the reaction space horizontally as plug flow;
mixing the biomass separately in each block in the reaction space and transferring biomass forward in the reaction space;
feeding the microbial strain of each block to a front of the block and, in at least two of the blocks, the feeding the microbial strain includes feeding eject obtained from the same block as high consistency stock; and
recovering biogas produced as a result of anaerobic digestion of said biomass in the reaction space.

22. The method according to claim 21, wherein the feeding the reject includes feeding the reject through mixing equipment.

23. The method according to claim 21, wherein dry content of the high consistency stock is 3% to 35%.

24. The method according to claim 21, wherein dry content of the high consistency stock is 10% to 20%.

25. The method according to claim 21, further comprising removing the reject from the blocks as a bottoms product utilizing a vacuum.

26. The method according to claim 25, further comprising generating said vacuum using a single pump and applying the vacuum in a selected block of a valve system.

27. The method according to claim 21, wherein the feeding the reject includes feeding the reject back to the respective block via a reject collection and feed system at regular intervals for keeping the reject collection and feed system clean.

28. The method according to claim 21, further comprising feeding the high consistency stock from at least one of walls of the reaction space and bottom of the reaction space to facilitate flow of biomass.

29. The method according to claim 21, further comprising feeding biogas from at least one of walls of the reaction space and bottom of the reaction space to facilitate flow of biomass.

30. The method according to claim 22, wherein the mixing equipment comprises rotating blade elements, the method further comprising operating the mixing equipment for the last block in a travel direction of biomass in a direction opposite to that of the mixing equipment of other blocks.

31. The method according to claim 22, further comprising inoculating the biomass by rotating the mixing equipment backwards at regular intervals.

32. A reactor for producing biogas from biomass using anaerobic digestion, comprising
a frame delimiting a reaction space for the biomass, the frame comprising a horizontal channel for plug flow of biomass that including at least three successive blocks containing respective microbial strains;
mixing equipment for mixing the biomass, feeding microbes to the biomass and transferring biomass forward in the reaction space;
a reject collection and feed system for collecting reject from the blocks and feeding the reject to the at least three blocks as high consistency stock through the mixing equipment, wherein biogas is generated from anaerobic digestion of the biomass by microbes of the respective microbial strains in the blocks; and
recovery equipment to recover the generated biogas.

33. The reactor according to claim 32, wherein the mixing equipment comprises shafts disposed transversely relative to a longitudinal direction of the frame of the reactor for mixing biomass in the respective blocks containing block-specific microbial strains.

34. The reactor according to claim 32, wherein the reject collection and feed system is arranged to feed the reject as high consistency stock through the mixing equipment to reduce friction.

35. The reactor according to claim 32, wherein the reject collection and feed system comprises a high consistency stock pump for feeding reject at a dry content of 3% to 35%.

36. The reactor according to claim 32, wherein the reject collection and feed system comprises a high consistency stock pump for feeding reject at a dry content of 10% to 20%.

37. The reactor according to claim 32, wherein the mixing equipment of each block includes support and operating elements located outside the frame.

38. The reactor according to claim 32, further comprising equipment for feeding at least one of high consistency stock and biogas from at least one of walls of the reaction space, and a bottom of the reaction space to the biomass in order to facilitate the flow.

39. The reactor according to claim 32, wherein the frame of the reactor comprises sub-frames identical with each other except for their lengths and heights.

40. The reactor according to claim 32, wherein the blocks of the reaction space are specific to a different one of the following reaction step of the anaerobic digestion:
a hydrolysis block,
an acid fermentation block
a methanogenesis block.

41. The reactor according to claim 35, wherein the high consistency stock pump comprises a hose pump.

42. The reactor according to claim 32, wherein the at least three blocks of the horizontal channel of the reaction space include three to six blocks.
